(19) United States
(12) Patent Application Publication
(10) Pub. No.: US 2007/0233256 A1
(43) Pub. Date: Oct. 4, 2007

(54) Facet and disc arthroplasty system and method

(76) Inventors: John Arthur Ohrt, Redmond, WA (US); Cin Abidin, Issaquah, WA (US); Michael J. Funk, North Bend, WA (US); Mark K. Kuiper, Seattle, WA (US); Anthony V. Finazzo, Lake Forest Park, WA (US)

Correspondence Address: SHAY LAW GROUP LLP, 2755 CAMPUS DRIVE, SUITE 210, SAN MATEO, CA, 94403, US

(21) Appl. No.: 11/724,927

(22) Filed: Mar. 15, 2007

Related U.S. Application Data
(60) Provisional application No. 60/782,932, filed on Mar. 15, 2006.

(51) Int. Cl. A61F 2/44

(52) U.S. Cl. 623/17.11; 606/61

(57) ABSTRACT

The invention discloses devices, methods and systems for implanting articulating devices on the human spine. In some embodiments, three or more vertebrae are movable interconnected with artificial joints. Some embodiments are particularly well suited for implanting in the lumbosacral area of the spine. Some embodiments employ hybrid systems combining translaminar fixation with pedicular fixation of the device components. Some embodiments combine facet joint replacement with disc replacement.

### CROSS-REFERENCE

This application claims the benefit of U.S. Provisional Patent Application No. 60/782,932 to Ohrt et al., filed Mar. 15, 2006, the disclosure of which is incorporated herein as if fully set forth.

### FIELD OF THE INVENTION

The present invention generally relates to devices and surgical methods for treatment of various spinal pathologies. More specifically, the present invention is directed to configurable and anatomically adaptable implantable devices for use in a spine and surgical procedures for altering the biomechanics of a spine, either temporarily or permanently. The devices alter, replace and/or revise existing anatomy and/or previously implanted devices.

### BACKGROUND OF THE INVENTION

Back pain, particularly in the small of the back, or lumbosacral region (L4-S1) of the spine (see, FIG. 1), is a common ailment. In many cases, the pain severely limits a person's functional ability and quality of life. Back pain interferes with work, routine daily activities, and recreation. It is estimated that Americans spend $50 billion each year on low back pain alone. It is the most common cause of job-related disability and a leading contributor to missed work.

Through disease or injury, the laminae, spinous process, articular processes, facets and/or facet capsules of one or more vertebral bodies along with one or more intervertebral discs can become damaged which can result in a loss of proper alignment or loss of proper articulation of the vertebra. This damage can result in an anatomical change, loss of mobility, and pain or discomfort. For example, the vertebral facet joints can be damaged by traumatic injury or as a result of disease. Diseases damaging the spine and/or facets include osteoarthritis where the cartilage of joints is gradually worn away and the adjacent bone is remodeled, ankylosing spondylolysis (or rheumatoid arthritis) of the spine which can lead to spinal rigidity, and degenerative spondylolisthesis which results in a forward displacement of the lumbar vertebra on the sacrum. Damage to facet joints of the vertebral body often results in pressure on nerves, commonly referred to as “pinched” nerves, or nerve compression or impingement. The result is pain, misaligned anatomy, a change in biomechanics and a corresponding loss of mobility. Pressure on nerves can also occur without facet joint pathology, e.g., as a result of a herniated disc.

One conventional treatment of facet joint pathology is spine stabilization, also known as intervertebral stabilization. Intervertebral stabilization desirably controls, prevents or limits relative motion between the vertebrae, through the use of spinal hardware, removal of some or all of the intervertebral disc, fixation of the facet joints, bone graft/osteo-inductive/osteo-conductive material positioned between the vertebral bodies (with or without concurrent insertion of fusion cages), and/or some combination thereof, resulting in the fixation of (or limiting the motion of) any number of adjacent vertebrae to stabilize and prevent/limit/control relative movement between those treated vertebrae.

Although spine fusion surgery is an efficacious treatment alternative, complications can, nonetheless, result. Patients undergoing spine surgery frequently continue to experience symptoms. For surgical procedures in the lumbar spine, failure rates as high as 37% have been reported after lumbar fusion and 30% for surgery without fusion. See Eichholz, et al., “Complications of Revision Spinal Surgery,” Neurosurg Focus 15(3): 1-4 (2003). Post-operative problems can include: decompression related problems, and fusion related problems. Decompression related problems (i.e., loss of normal spine balance resulting in the head and trunk no longer being centered over the pelvis) include, for example, recurrent disc herniation, spinal stenosis, chronic nerve injury, infection, and decompression. Fusion related problems can include, pain from the bone harvest site, failure of a fusion to develop, loosening of the implanted devices, nerve irritation caused by the devices, infection, and poor alignment of the spine.

Stabilization of vertebral bodies can also be achieved (to varying degrees) from a wide variety of procedures, including the insertion of motion limiting devices (such as intervertebral spacers, artificial ligaments and/or dynamic stabilization devices), devices promoting arthrodesis (rod and screw systems, cables, fusion cages, etc.), and complete removal of some or all of a vertebral body from the spinal column (which may be due to extensive bone damage and/or tumorous growth inside the bone) and insertion of a vertebral body replacement (generally anchored into the adjacent upper and lower vertebral bodies). Various devices are known for fixing the spine and/or sacral bone adjacent the vertebra, as well as attaching devices used for fixation, including devices disclosed in: U.S. Pat. Nos. 6,585,769; 6,290,703; 5,782,833; 5,738,585; 6,547,790; 6,638,321; 6,520,963; 6,074,391; 5,569,247; 5,891,145; 6,090,111; 6,451,021; 5,683,392; 5,863,293; 5,964,760; 6,010,503; 6,019,759; 6,540,749; 6,077,262; 6,248,105; 6,524,315; 5,797,911; 5,879,350; 5,885,285; 5,643,263; 6,565,565; 5,725,527; 6,471,705; 6,554,843; 5,575,792; 5,688,274; 5,690,630; 6,022,350; 4,805,602; 5,474,555; 4,611,581; 5,129,900; 5,741,255; 6,132,430; and U.S. Patent Publication Nos. 2002/0120272, 2005/0143818, 2005/0240265, 2005/0240266, 2006/0058791 and 2006/0149375.

More recently, various treatments have been proposed and developed as alternatives to spinal fusion. Many of these treatments seek to restore (and/or maintain) some, or all, of the natural motion of the treated spinal unit, and can include intervertebral disc replacement, nucleus replacement, facet joint resurfacing, and facet joint replacement. Such solutions typically include devices that do not substantially impair spinal movement. See, U.S. Pat. Nos. 6,610,091; 6,811,567; 6,902,580; 5,571,171; and Re 36,758; and PCT Publication Nos. WO 01/158563, WO 2004/103228, WO 2005/009301, and WO 2004/103227. Thus, spinal arthroplasty has become an acceptable alternative to fusion, particularly in cases of degenerative disc disease. Arthroplasty devices can be particularly useful because the devices are designed to create an artificial joint or restore the functional integrity and power of a joint.

### SUMMARY OF THE INVENTION

It is being discovered that spinal arthroplasty methods and devices that are suitable for use at various levels of the spine do not perform adequately at other levels of the spine. For example, implantable devices that work well at replacing the facet joints at level T11-T12 or L1-L2 may or may not perform adequately at levels L4-L5 or L5-S1. What is needed are devices that can reliably be used at specific levels of the spine, particularly multiple adjacent levels.

For the sake of description herein, the tools and prostheses that embody features of the invention are identified as either “cephalad” or “caudal” with relation to the portion of a given natural facet joint they replace. As previously described, a natural facet joint, such as facet joint 32 (FIG. 3), has a superior half and an inferior half. In anatomical terms, the superior half of the joint is formed by the vertebral level below the joint, which can thus be called the “caudal” portion of the facet joint because it is closer to the feet of the person. The inferior half of the facet joint is formed by the vertebral level above the joint, which can thus be called the “cephalad” portion of the facet joint because it is closer to the head of the person. Thus, the prosthesis that is used in the replacement of the caudal portion of a natural facet joint (i.e., the superior half) will be called a “caudal” prosthesis. Likewise, the prosthesis that is used in the replacement of the cephalad portion of a natural facet joint (i.e., the inferior half) will be called a “cephalad” prosthesis.

In certain patients, it may be desirable to replace the natural facet joints at more than one level. According to aspects of the invention, when facets joints are being replaced at two different levels, particularly if they are adjacent levels, a single implantable device, or multiple devices sharing one or more common components may be utilized. In such an embodiment, a portion of the device may serve as both a “caudal” prosthesis (to replace a lower portion of the facet joint located above the device) and a “cephalad” prosthesis (to replace an upper portion of the facet joint located below the device.

The invention relates to an implantable spinal arthroplasty devices and methods for their use. Some embodiments of the invention include a device with a first portion adapted to engage a vertebra at the L5 level of the spine and second portion adapted to engage a portion of a sacrum. The first and second portions of the device, per embodiments of the invention, cooperate to form at least one artificial facet joint between the L5 level and the sacrum.

The vertebra has two pedicles, a lamina, and a spinous process. In some embodiments of the device, the first portion of the device is configured to engage the pedicles of the L5 vertebra. In some of these embodiments, first portion includes a first attachment member and a second attachment member, and each attachment member is configured to connect to one of the pedicles, the first portion further comprising a cross arm spanning between the first and the second attachment members.

In some embodiments of the device, the first portion is configured to engage the lamina of the L5 vertebra. In some of these embodiments the first portion is configured to hook over an upper surface of the lamina.

In some embodiments of the device, the first portion includes two generally vertical members configured to be located on laterally opposite sides of a spinal process at the L5 level as well as a cross member configured to interconnect the generally vertical members and to be located beneath the spinal process.

In some embodiments of the device, the first portion includes at least one threaded fastener configured to penetrate the lamina. In other embodiments, the first portion includes at least one threaded fastener configured to press against an outer surface of the lamina.

Some embodiments of the device further include a joint interconnecting the first and the second portions of the device, the joint have a generally ball-shaped portion and a cup portion, wherein the ball-shaped portion may slide relative to the cup portion.

Embodiments of the invention include a method of implanting embodiments of the above-summarized spinal arthroplasty device; the method includes the attaching the device to the spine of a patient. The method may further include replacing at least a portion of a natural disc located adjacent to L5 vertebra with an artificial disc implant.

Embodiments of the invention also include a multi-level implantable spinal arthroplasty device with a first portion connectable to a first vertebra, a second portion connectable to a second vertebra located beneath the first vertebra, and a third portion connectable to a third vertebra located beneath the second vertebra. This embodiment includes at least one upper artificial joint interconnecting and allowing relative movement between the first and the second portions, and it includes at least one lower artificial joint interconnecting and allowing relative movement between the second and the third portions.

In some embodiments of this multi-level device, the first, the second, and third vertebrae are adjacent to one another. In some of these embodiments the first and second vertebrae are at the L4 and L5 levels, respectively, and the third vertebra is part of the sacrum. In some of the multi-level device embodiments, the first portion is configured to attach to a lamina of the first vertebra. In some of the multi-level device embodiments, the second portion is configured to attach to at least one pedicle of the second vertebra. In some of the multi-level device embodiments, the third portion is configured to attach to at least one pedicle of the third vertebra. In some of the multi-level device embodiments, the first portion is configured to attach to a lamina of the first vertebra and the second portion is configured to attach to at least one pedicle of the second vertebra; and in some such embodiments, the third portion is configured to attach to the sacrum.

In some embodiments of the multi-level device, the first portion includes two elongated bar elements, each element configured to pass diagonally through a lamina of the first vertebra. In some of these embodiments, at least one upper artificial joint includes a convex member and a concave member, the two members being configured to inter-engage to provide relative movement between the first and the second portions, one of the two members being located on a lower end of one of the elongated bar elements. In some of these embodiments, the at least one lower artificial joint includes a generally ball-shaped portion and a cup portion, wherein the ball-shaped portion may slide relative to the cup portion.

In embodiments of the multi-level device, the second portion includes a first attachment member and a second attachment member, each attachment member configured to connect to a pedicle of the second vertebra, the second portion further including a cross arm spanning between the first and the second attachment members.

Embodiments of the invention include a method of implanting embodiments of the above-summarized multi-level spinal arthroplasty device; the method includes attaching the device of claim B1 to the spine of a patient. In some embodiments of this method, the multi-level device is attached to three adjacent vertebrae. In some embodiments, the first vertebra is at the L4 level, the second vertebra is at the L5 level, and the third vertebra is part of the sacrum. The method may further include replacing at least a portion of a natural disc adjacent to any of the first, the second, and/or third vertebrae with an artificial disc implant.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a lateral elevation view of a normal human spinal column;

FIG. 2 is a superior view of a normal human lumbar vertebra;

FIG. 3 is a lateral elevational view of two vertebral bodies forming a functional spinal unit;

FIG. 4 is a posterolateral oblique view of a vertebrae from a human spinal column;

FIG. 5 is a perspective view of the anatomical planes of the human body;

FIG. 6 is a posterior oblique view of an implantable spinal arthroplasty device;

FIG. 7 is a posterior elevational view of an implantable spinal arthroplasty device;

FIG. 8 is an oblique partial cutaway view of the device of FIG. 7;

FIG. 9 is an exploded view of a portion of the device of FIG. 7;

FIG. 10 is a lateral elevational view of a translaminar cephalad anchoring device implanted in a vertebra;

FIG. 11 is a partially exploded lower plan view of the device of FIG. 10;

FIG. 12 is an oblique cross-sectional view of the device of FIG. 10;

FIG. 13 is an oblique cross-sectional view of another translaminar cephalad anchoring device implanted in a vertebra;

FIG. 14 is an oblique cross-sectional view of yet another translaminar cephalad anchoring device implanted in a vertebra;

FIG. 15 is a posterior elevational view of an arthroplasty device shown implanted on an L5 vertebra and a sacrum;

FIG. 16 is a posterolateral oblique view of the device of FIG. 15 before implantation;

FIG. 17 is a posterior elevational view of an arthroplasty device shown implanted on an L5 vertebra and a sacrum;

FIG. 18 is a posterolateral oblique view of a caudal anchoring device implanted on a sacrum;

FIG. 19 is a perspective view of a portion of the device of FIG. 18;

FIG. 20 is an oblique side view of one embodiment of a caudal bearing cup;

FIG. 21 is an oblique side view of another embodiment of a caudal bearing cup;

FIG. 22 is a posterior elevational view of an arthroplasty device shown implanted on an L5 vertebra and a sacrum;

FIGS. 23 is a posterior elevational view of a hybrid, multilevel arthroplasty device shown implanted on L4 and L5 vertebrae and a sacrum;

FIG. 24 is a posterolateral oblique view of another hybrid, multilevel arthroplasty device shown implanted on L4 and L5 vertebrae and a sacrum; and

FIG. 25 is a posterolateral oblique view of yet another hybrid, multilevel arthroplasty device shown implanted on L4 and L5 vertebrae and a sacrum.

### DETAILED DESCRIPTION OF THE INVENTION

The invention relates generally to implantable devices, apparatus or mechanisms that are suitable for implantation within a human body to restore, augment, and/or replace soft tissue and connective tissue, including bone and cartilage, and systems for treating spinal pathologies. In some instances the implantable devices can include devices designed to replace missing, removed or resected body parts or structure. The implantable devices, apparatus or mechanisms are configured such that the devices can be formed from parts, elements or components which alone or in combination comprise the device. The implantable devices can also be configured such that one or more elements or components are formed integrally to achieve a desired physiological, operational or functional result such that the components complete the device. Functional results can include the surgical restoration and functional power of a joint, controlling, limiting or altering the functional power of a joint, and/or eliminating the functional power of a joint by preventing joint motion. Portions of the device can be configured to replace or augment existing anatomy and/or implanted devices, and/or be used in combination with resection or removal of existing anatomical structure.

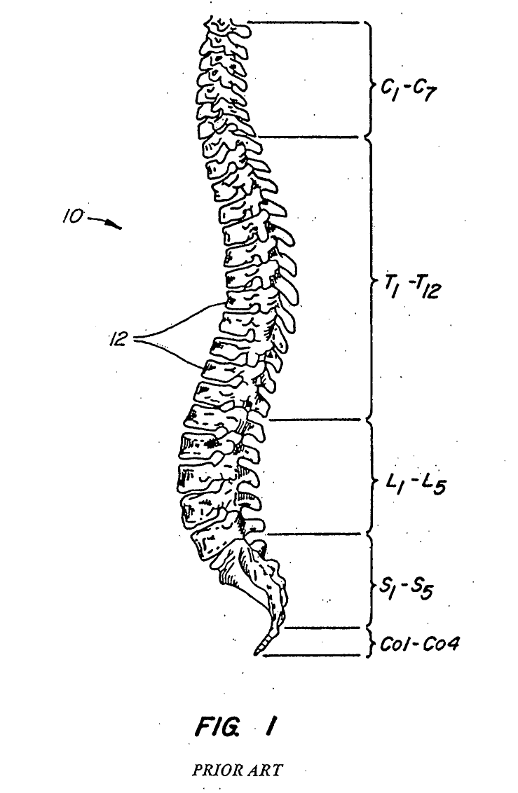

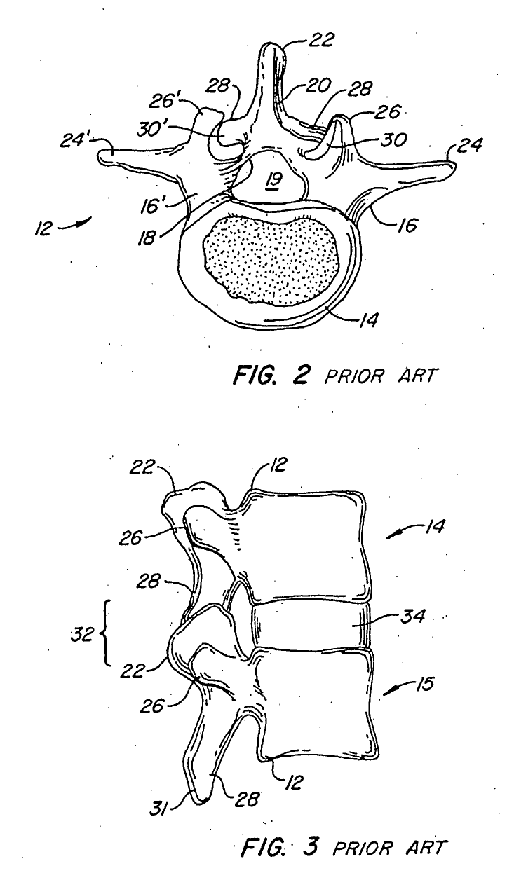

The devices of the invention are designed to interact with the human spinal column 10, as shown in FIG. 1, which is comprised of a series of thirty-three stacked vertebrae 12 divided into five regions. The cervical region includes seven vertebrae, known as C1-C7. The thoracic region includes twelve vertebrae, known as T1-T12. The lumbar region contains five vertebrae, known as L1-L5. The sacral region is comprised of five fused vertebrae, known as S1-S5, while the coccygeal region contains four fused vertebrae, known as Co1-Co4. An example of one of the vertebra is illustrated in FIG. 2 which depicts a superior plan view of a normal human lumbar vertebra 12. Although human lumbar vertebrae vary somewhat according to location, the vertebrae share many common features. Each vertebra 12 includes a vertebral body 14. Two short boney protrusions, the pedicles 16, 16′, extend dorsally from each side of the vertebral body 14 to form a vertebral arch 18 which defines the vertebral foramen 19. At the posterior end of each pedicle 16, the vertebral arch 18 flares out into broad plates of bone known as the laminae 20. The laminae 20 fuse with each other to form a spinous process 22. The spinous process 22 provides for muscle and ligamentous attachment. A smooth transition from the pedicles 16 to the laminae 20 is interrupted by the formation of a series of processes.

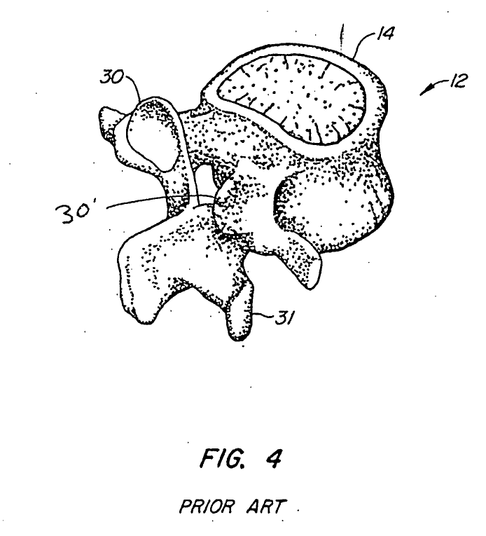

Two transverse processes 24, 24′ thrust out laterally, one on each side, from the junction of the pedicle 16 with the lamina 20. The transverse processes 24, 24′ serve as levers for the attachment of muscles to the vertebrae 12. Four articular processes, two superior 26, 26′ and two inferior 28, 28′, also rise from the junctions of the pedicles 16 and the laminae 20. The superior articular processes 26, 26′ are sharp oval plates of bone rising upward on each side of the vertebrae, while the inferior processes 28, 28′ are oval plates of bone that jut downward on each side. See also FIG. 4.

The superior and inferior articular processes 26 and 28 each have a natural bony structure known as a facet. The superior articular facet 30 faces medially upward, while the inferior articular facet 31 (see FIG. 3) faces laterally downward. When adjacent vertebrae 12 are aligned, the facets 30, 31, which are capped with a smooth articular cartilage and encapsulated by ligaments, interlock to form a facet joint 32. The facet joints are apophyseal joints that have a loose capsule and a synovial lining.

As discussed, the facet joint 32 is comprised of a superior facet and an inferior facet (shown in FIG. 4). The superior facet is formed in the vertebral level below the joint 32, and the inferior facet is formed in the vertebral level above the joint 32. For example, in the L4-L5 facet joint shown in FIG. 3, the superior facet of the joint 32 is formed by bony structure on the L5 vertebra (i.e., a superior articular surface and supporting bone 26 on the L5 vertebra), and the inferior facet of the joint 32 is formed by bony structure on the L4 vertebra (i.e., an inferior articular surface and supporting bone 28 on the L4 vertebra). The angle formed by a facet joint located between a superior facet and an inferior facet changes with respect to the midline depending upon the location of the vertebral body along the spine. The facet joints do not, in and of themselves, substantially support axial loads unless the spine is in an extension posture (lordosis). As would be appreciated by those of skill in the art, the orientation of the facet joint for a particular pair of vertebral bodies changes significantly from the thoracic to the lumbar spine to accommodate a joint's ability to resist flexion-extension, lateral bending, and rotation.

An intervertebral disc 34 located between each adjacent vertebra 12 (with stacked vertebral bodies shown as 14, 15 in FIG. 3) permits gliding movement between the vertebrae 12. The structure and alignment of the vertebrae 12 thus permit a range of movement of the vertebrae 12 relative to each other. FIG. 4 illustrates a posterolateral oblique view of a vertebrae 12, further illustrating the curved surface of the superior articular facet 30 and the protruding structure of the inferior facet 31 adapted to mate with the opposing superior articular facet. As discussed above, the position of the inferior facet 31 and superior facet 30 varies on a particular vertebral body to achieve the desired biomechanical behavior of a region of the spine.

Thus, overall the spine comprises a series of functional spinal units that are a motion segment consisting of two adjacent vertebral bodies, the intervertebral disc, associated ligaments, and facet joints. See Posner, I, et al. “A biomechanical analysis of the clinical stability of the lumbar and lumbosacral spine.” Spine 7:374-389 (1982).

As previously described, a natural facet joint, such as facet joint 32 (FIG. 3), has a superior facet 30 and an inferior facet 31. In anatomical terms, the superior facet of the joint is formed by the vertebral level below the joint, which can thus be called the “caudal” portion of the facet joint because it is anatomically closer to the tail bone or feet of the person. The inferior facet of the facet joint is formed by the vertebral level above the joint, which can be called the “cephalad” portion of the facet joint because it is anatomically closer to the head of the person. Thus, a device that, in use, replaces the caudal portion of a natural facet joint (i.e., the superior facet 30) can be referred to as a “caudal” device. Likewise, a device that, in use, replaces the cephalad portion of a natural facet joint (i.e., the inferior facet 31) can be referred to a “cephalad” device.

When the processes on one side of a vertebral body 14 are spaced differently from processes on the other side of the same vertebral body, components of the devices on each side would desirably be of differing sizes as well to account for anatomical difference that can occur between patients. Moreover, it can be difficult for a surgeon to determine the precise size and/or shape necessary for an implantable device until the surgical site has actually been prepared for receiving the device. In such case, the surgeon typically can quickly deploy a family of devices possessing differing sizes and/or shapes during the surgery. Thus, embodiments of the spinal devices of the present invention include modular designs that are either or both configurable and adaptable. Additionally, the various embodiments disclosed herein may also be formed into a “kit” or system of modular components that can be assembled in situ to create a patient specific solution. As will be appreciated by those of skill in the art, as imaging technology improves, and mechanisms for interpreting the images (e.g., software tools) improve, patient specific designs employing these concepts may be configured or manufactured prior to the surgery. Thus, it is within the scope of the invention to provide for patient specific devices with integrally formed components that are pre-configured.

A configurable modular device design, such as the one enabled by this invention, allows for individual components to be selected from a range of different sizes and utilized within a modular device. One example of size is to provide caudal and cephalad stems of various lengths. A modular implantable device design allows for individual components to be selected for different functional characteristics as well. One example of function is to provide stems having different surface features and/or textures to provide anti-rotation capability. Other examples of the configurability of modular implantable device of the present invention as described in greater detail below.

Implantable devices of the present invention are configurable such that the resulting implantable spinal device is selected and positioned to conform to a specific anatomy or desired surgical outcome. The adaptable aspects of embodiments of the present invention provide the surgeon with customization options during the implantation or revision procedure. It is the adaptability of the present device systems that also provides adjustment of the components during the implantation procedure to ensure optimal conformity to the desired anatomical orientation or surgical outcome. An adaptable modular device of the present invention allows for the adjustment of various component-to-component relationships. One example of a component-to-component relationship is the rotational angular relationship between a crossbar mount and the crossbar. Other examples of the adaptability of modular device of the present invention as described in greater detail below. Configurability may be thought of as the selection of a particular size of component that together with other component size selections results in a “custom fit” implantable device. Adaptability then can refer to the implantation and adjustment of the individual components within a range of positions in such a way as to fine tune the “custom fit” devices for an individual patient. The net result is that embodiments of the modular, configurable, adaptable spinal device and systems of the present invention allow the surgeon to alter the size, orientation, and relationship between the various components of the device to fit the particular needs of a patient during the actual surgical procedure.

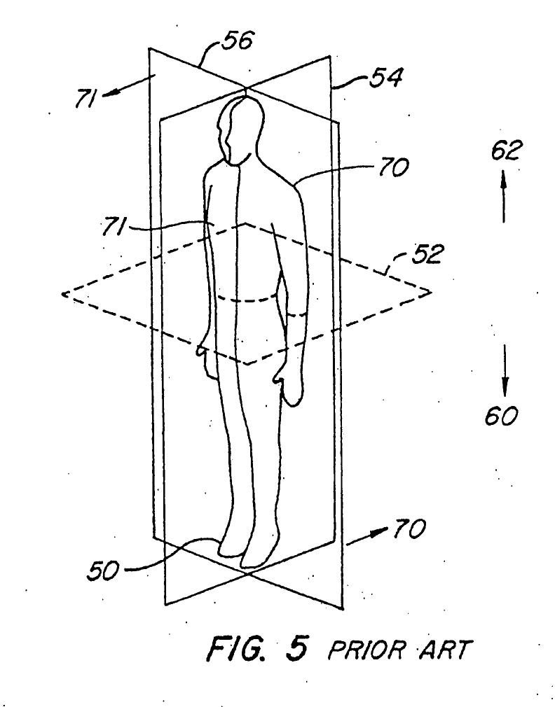

In order to understand the configurability, adaptability and operational aspects of the invention, it is helpful to understand the anatomical references of the body 50 with respect to which the position and operation of the devices, and components thereof, are described. There are three anatomical planes generally used in anatomy to describe the human body and structure within the human body: the axial plane 52, the sagittal plane 54 and the coronal plane 56 (see FIG. 5). Additionally, devices and the operation of devices are better understood with respect to the caudal 60 direction and/or the cephalad direction 62. Devices positioned within the body can be positioned dorsally 70 (or posteriorly) such that the placement or operation of the device is toward the back or rear of the body. Alternatively, devices can be positioned ventrally 71 (or anteriorly) such that the placement or operation of the device is toward the front of the body. Various embodiments of the spinal devices and systems of the present invention may be configurable and variable with respect to a single anatomical plane or with respect to two or more anatomical planes. For example, a component may be described as lying within and having adaptability in relation to a single plane. For example, a stem may be positioned in a desired location relative to an axial plane and may be moveable between a number of adaptable positions or within a range of positions. Similarly, the various components can incorporate differing sizes and/or shapes in order to accommodate differing patient sizes and/or anticipated loads.

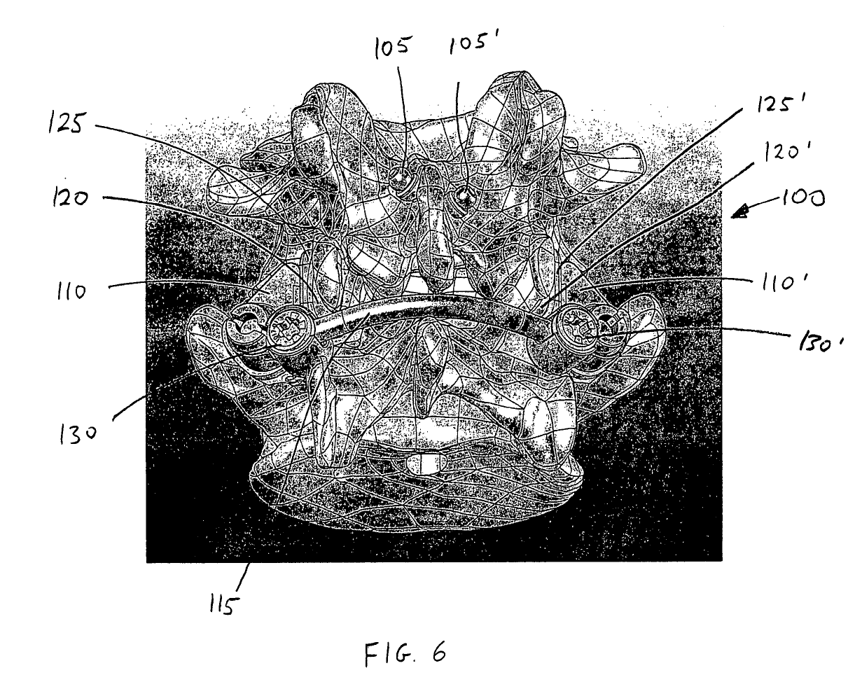

Turning now to FIG. 6, an isometric view of a modular, configurable and adaptable implantable spinal arthroplasty device 100 is depicted. The spinal arthroplasty device 100 is illustrated implanted into vertebral bodies 14.

The arthroplasty device 100 and the various other devices disclosed herein can be formed of a variety of materials. For example, where the devices have bearing surfaces (i.e. surfaces that contact another surface), the surfaces may be formed from biocompatible metals such as cobalt chromium steel, surgical steel, titanium, titanium alloys, tantalum, tantalum alloys, aluminum, etc. Suitable ceramics and other suitable biocompatible materials known in the art can also be used. Suitable polymers include polyesters, aromatic esters such as polyalkylene terephthalates, polyamides, polyalkenes, poly(vinyl) fluoride, PTFE, polyarylethyl ketone, and other materials that would be known to those of skill in the art. Various alternative embodiments of the spinal arthroplasty device could comprise a flexible polymer section (such as a biocompatible polymer) that is rigidly or semi rigidly fixed to the adjacent vertebral bodies whereby the polymer flexes or articulates to allow the vertebral bodies to articulate relative to one another.

The spinal arthroplasty device 100 includes a pair of cephalad translaminar anchors 105, 105′ and a pair of caudal pedicle anchors 110, 110′. The caudal pedicle anchors 110, 110′ are supplemented with a crossbar 115. In this exemplary embodiment, translaminar anchors 105, 105′ each support a spherical cephalad bearing surface 120, 120′ mounted on their lower ends and held flush to the cephalad facet. Pedicle anchors 110, 110′ each support a concave caudal bearing surface 125, 125′ adjacent to a cephalad bearing surface 120, 120′. In this embodiment the natural facet joints of the spine (FIG. 3, 32) are replaced by the cooperative metal-on-metal (e.g. cobalt chromium) operation of the cephalad bearing surfaces 120, 120′ with the caudal bearing surfaces 125, 125′. The components of the spinal facet arthroplasty device 100 are designed to provide appropriate configurability and adaptability for the given disease state, patient specific anatomy and spinal level where the implant occurs.

Each end of the crossbar 115 may be mounted to a caudal pedicle anchor 110, 110′ with a multi-axis tulip 130, 130′. A crossbar 115 may be selected from a variety of straight, curved or complex shaped crossbars depending on the particular application and anatomy of the patient.

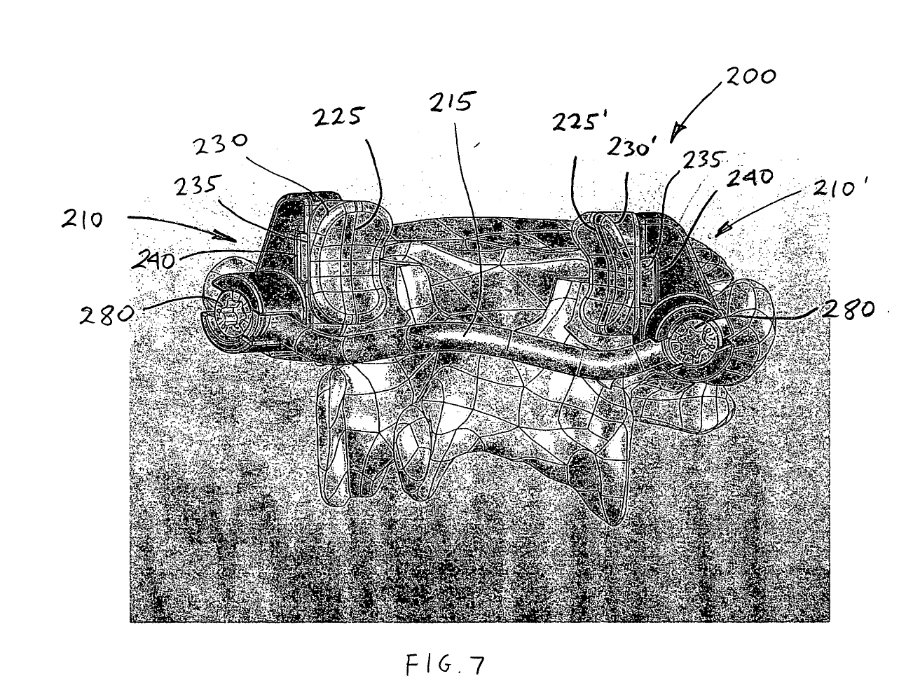

Another implantable arthroplasty device 200 is illustrated in FIGS. 7-9. FIG. 7 shows the artificial joint structure mounted to the pedicles of a vertebra for replacing the natural facets (see FIG. 4, 30 and 30′). The caudal structure 200 is designed to mate with a cephalad structure or structures, similar to the embodiment shown in FIG. 6. Caudal structure 200 includes caudal pedicle anchors 210, 210′. The caudal pedicle anchors 210, 210′ are supplemented with a crossbar 215. In this embodiment, crossbar 215 includes three bends to avoid adjacent spinal anatomy. Pedicle anchors 210, 210′ each support a concave caudal bearing surface 225, 225′.

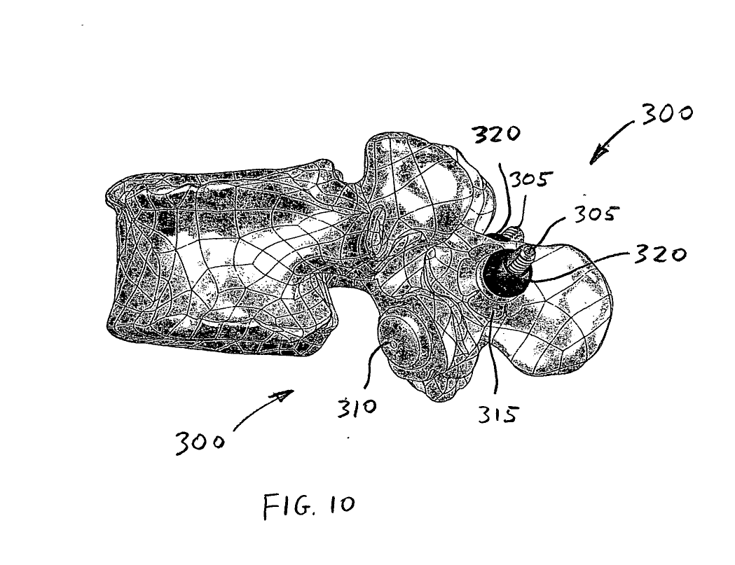

As best seen in FIG. 9, caudal bearing surfaces 225, 225′ are formed on modular bearing elements 230, 230′. Modular bearing elements 230, 230′ in turn are connected to pedicle anchors 210, 210′, such as with mating tapered dovetail surfaces 235, 240 as shown.

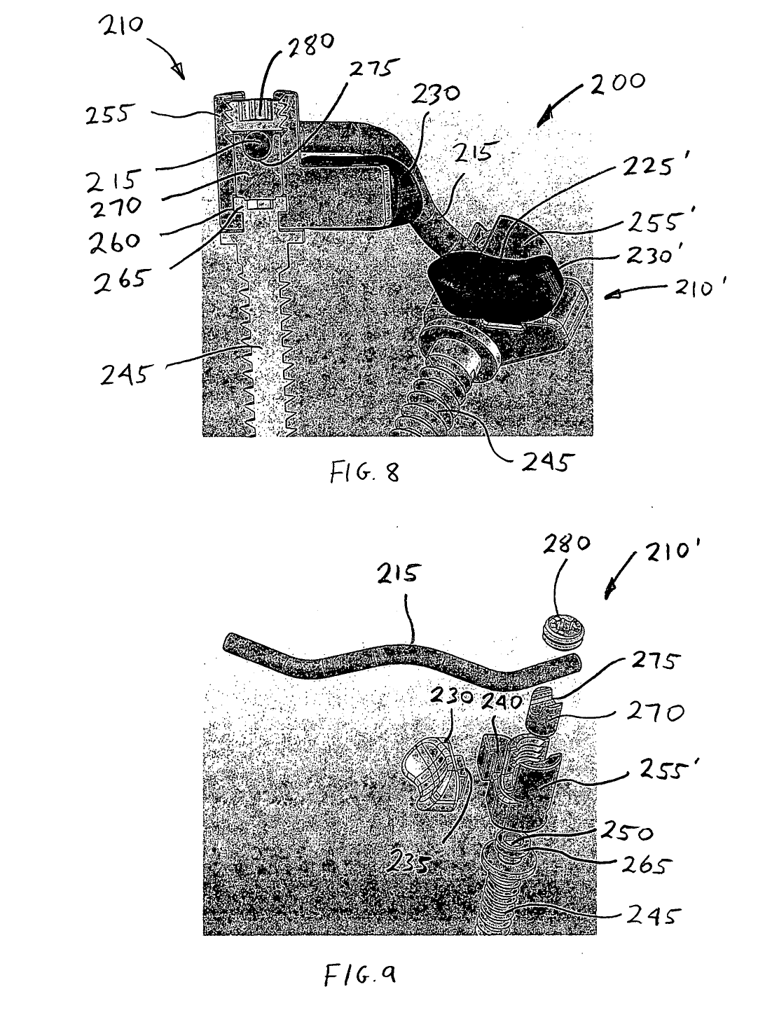

As shown in FIGS. 8 and 9, pedicle anchors 210, 210′ are configured to be mounted to lamina pedicles with pedicle screws 245. Pedicle screws 245 include a driver portion 250 to allow the screw to be rotatably driven into the vertebra with a mating driving tool (not shown). Once pedicle screw 245 is placed in the vertebra, pedicle anchor body 255 may be slidably attached to the head of screw 245, such as by a T-shaped slot 260 in body 255 inter-engaging with a flange 265 on the screw head as shown. Crossbar lock 270 may then be placed in the bore of pedicle anchor body 255. Crossbar lock 270 may include a groove 275 formed in one end for receiving crossbar 215. The entire pedicle anchor assembly 210 may be secured by inserting threaded fastener 280 in the bore of pedicle anchor body 255 over crossbar 215 and tightening it down. As fastener 280 is turned in the threaded upper portion of the bore of body 255, fastener 280 bears down on crossbar 215. Crossbar 215 in turn bears down on crossbar lock 270, which bears down on the head of screw 245, thereby locking the crossbar 215, anchor body 255, and bearing element 230 onto the pedicle screw 245.

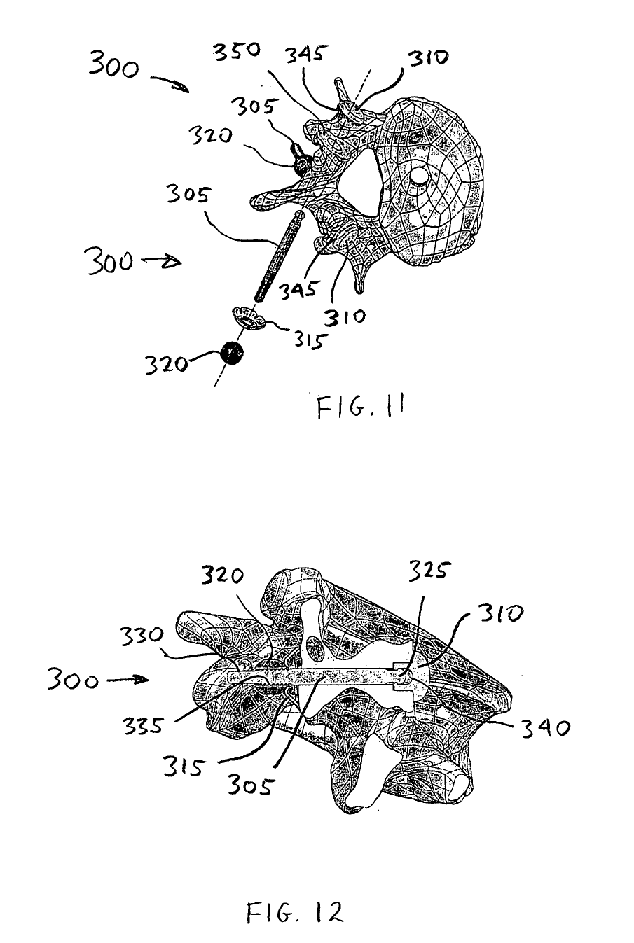

Referring now to FIGS. 10-12, another embodiment of translaminar cephalad anchors 300 is shown. Each anchor 300 comprises a pin 305, a cap 310, a spring washer 315 and a nut 320. Pin 305 is provided with a knob portion 325 at one end and a first threaded portion 330 and a second threaded portion 335 adjacent an opposite end. Cap 310 includes a recess 340 for receiving the knob portion 325 of pin 305. Cap 310 can lock onto knob portion 325 by utilizing a snap fit or by having a key-hole shaped lateral access channel in the cap that the knob portion 325 may slide into. Such an arrangement allows some angular misalignment between cap 310 and pin 305. In some embodiments, cap 310 is provided with teeth 345 around its outer periphery, as shown in FIG. 11, to allow cap 310 to better grip the outer surface of the lamina.

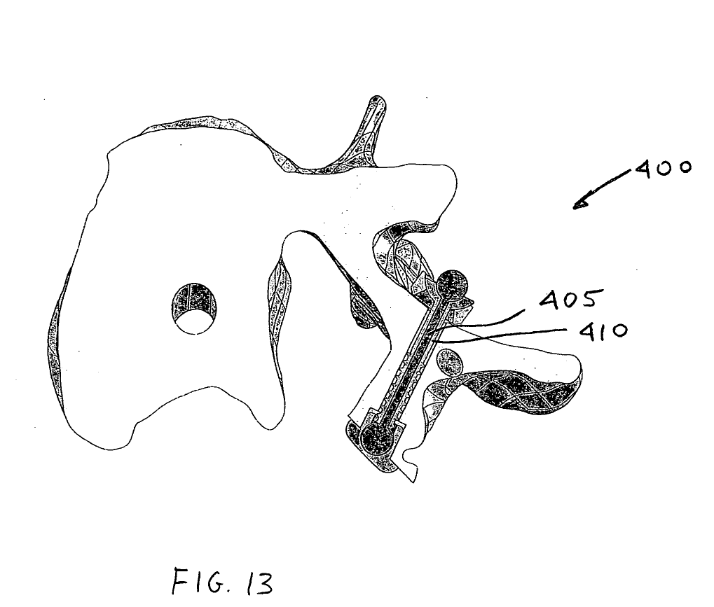

To install anchors 300 in a vertebra, the inferior facets are removed to leave a generally flat mounting surface 350 as shown. Transverse holes are drilled in the lamina as shown to receive pins 305. A counterbore may also be made in the lower side of each hole to receive a portion of cap 310, as best seen in FIG. 12. Cap 310 may be connected to the knob portion 325 of a pin 305 before it is inserted into the hole. Spring washer 315 may then be placed over the portion of pin 305 protruding from the opposite end of the hole and secured by tightening nut 320 on the second threaded portion 335 of pin 305 until spring washer 315 is snug against the bone.

Spring washer 315 is configured in a petal design to allow the washer edges to conform to an irregular bone surface. The springiness of the washer desirably creates tension which better secures the device to the bone. The washer can be used at any location where a device is adapted to engage bone and is suitable for use with any of the embodiments disclosed herein. The washer may also incorporate a textured or bony in-growth surface to facilitate bony fixation.

Nut 320 may be provided with a spherical surface on one or both sides as shown. Such a spherical surface may engage with spring washer 315 to allow the washer to better conform to a bone surface that is not completely perpendicular to pin 305. In this embodiment, once nut 320 is tightened against spring washer 315, the first threaded portion of pin 305 is exposed to allow additional hardware to be attached to anchor 300, if desired. Cap 310 itself may serve as a cephalad bearing surface for creating an artificial facet joint, or it may be used to secure other such hardware.

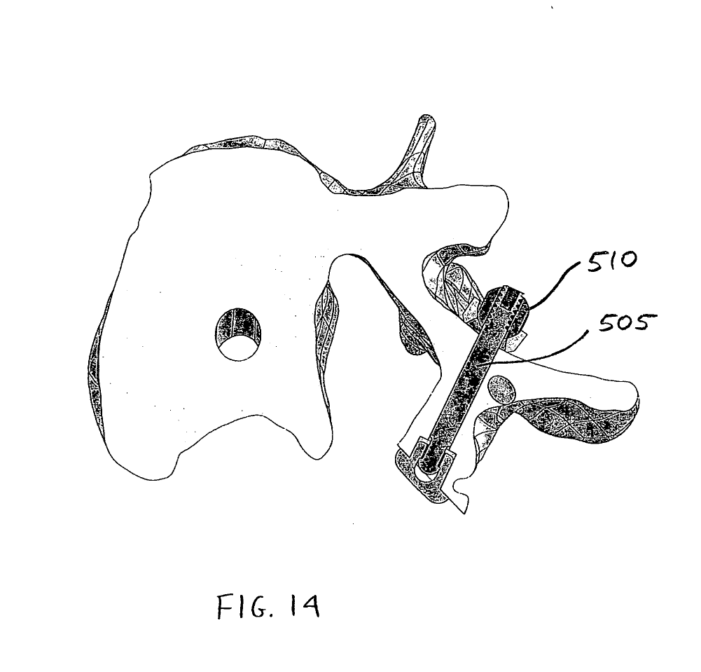

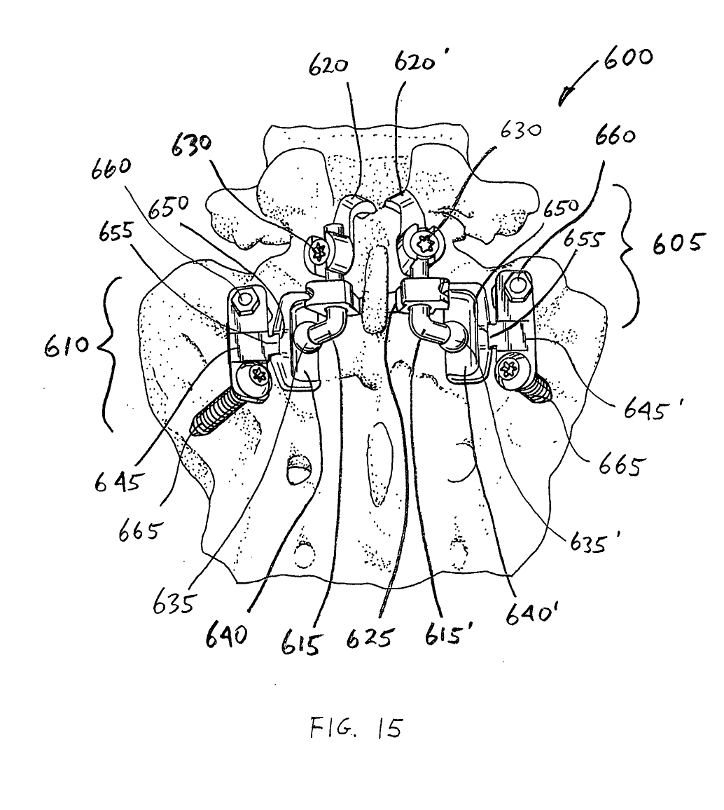

Referring now to FIG. 13, another embodiment of a cephalad anchor 400 is shown. In this embodiment, the fixation element comprises a cable 405 and cannulated tube 410, with the cannulated tube passing through the targeted bony structure of the lamina and/or spinous process and encircling the cable along at least a portion of its length. The cable 405 would desirably facilitate flexibility of the implanted artificial facet joint, while the cannulated tube would, among other things, significantly increase the cross-sectional surface area of the cable, thereby reducing the opportunity of the cable to “pull-out” of the bone laterally. A bearing engages one end of the cable and an anchor engages the opposing end. Alternatively, the fixation element may comprise a solid rod 505 having a flexible or adjustable engagement member 510 (see FIG. 14) allowing for some variation between the orientation of the cephalad bearing and the longitudinal axis of the rod. In these embodiments, the cross-section of the cable/rod is desirably of a sufficient size to prevent the force applied by the cable in the lamina from exceeding the ability of the lamina to resist the force; i.e., a cable of insufficient diameter could present a force great enough to tear through the lamina. Moreover, the cross-section of the cable/rod need not be circular, but may be any shape, including irregular shapes, to desirably present a surface to the surrounding bone that is (1) large enough (and/or flat enough) to resist “pull-out” through the bone (e.g. along the length of the cable), (2) non-circular to resist rotation of the cable/rod, and/or (3) of increased surface area to facilitate bony in-growth into the cable/rod. The anchors can further be adapted to provide internal threads such that the anchors can be secured to the cable by screwing the anchor onto a threaded end of the cable, or can be adapted to provide an aperture that enables the anchor to be snap fit onto the end of the cable. Other configurations will be apparent to those skilled in the art.

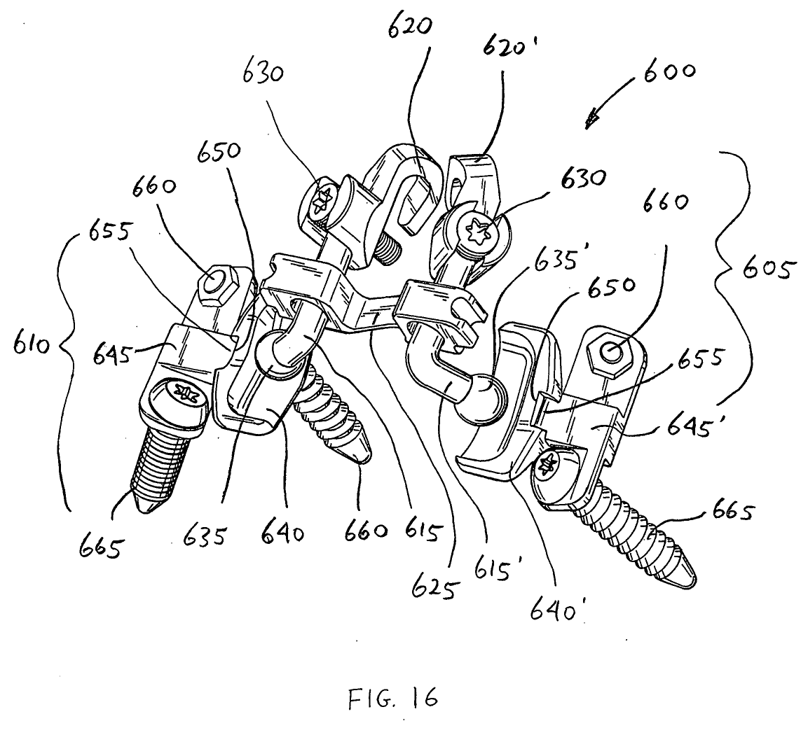

Referring now to FIGS. 15 and 16, isometric views of an implantable spinal arthroplasty device 600 are depicted with the device attached to the L5 level and sacrum of a spine (FIG. 15), and with the device shown by itself (FIG. 16). At certain levels of the spine, such as in the lumbosacral region, devices and methods of attaching them that work well at other levels of the spine may not work well or at all. For example, the thickness and/or orientation of a lamina in the lumbosacral region may not be sufficient to support a translaminar cephalad anchor as described above. Accordingly, new devices and attachment methods may be needed when attaching arthroplasty devices in the lumbosacral region.

Device 600 comprises a cephalad portion 605 and a caudal portion 610. In this embodiment, the cephalad portion 605 includes two arms 615, 615′ that have a hooked portion 620, 620′ on their upper ends that engage the upper edge of the lamina (such as the lamina of L5 as shown). When device 600 is implanted as depicted in FIG. 15, arms 615, 615′ reside on opposite sides of the spinous process, and in this embodiment are inter-connected by a crossbar 625 which engages the underside of the spinous process. Crossbar 625 may be secured to arms 615, 615′ by clamp screws, set screws, crimping, adhesive, or other methods known to those skilled in the art. Crossbar 625 may alternatively be formed from a plurality of pieces and/or formed integrally with arms 615, 615′. A variety of crossbar and/or arm configurations may be provided in the operating room such as in kit form to allow a custom cephalad portion 605 to be created to suit a particular application and/or anatomy of each patient.

Mounting screws 630 may be located on arms 615, 615′ for further securing cephalad portion 605 of device 600. Screws 630 may be configured to penetrate the lamina to hold arms 615, 615′ down against the lamina. Alternatively, screws 630 may be configured with flat tips as shown to press down against the lamina, thereby securing cephalad portion 605 on the vertebra by driving the distal regions of hooked portions 620, 620′ against the underside of the lamina and the crossbar 625 against the underside of the spinous process.

In this embodiment, the ends of arms 615, 615′ opposite the hooked portions 620, 620′ are bent laterally outward and anteriorly, and comprise spherical cephalad bearing elements 635, 635′. Bearing elements 635, 635′ are slidably received in caudal cups 640, 640′ which are part of the caudal portion 610 of device 600. In the embodiment shown, caudal cups 640, 640′ are separate elements from caudal anchor bases 645, 645′ to further increase the modularity of implantable device 600. Caudal cups 640, 640′ may be selected from a variety of different caudal cups to suit the particular application and/or patient anatomy, and may be attachable to the caudal anchor bases 645, 645′ by interlocking surfaces 650 and 655. Caudal anchor bases 645, 645′ may be attached to one or more vertebra, such as S1 as shown, with anchor screws 660 and 665. Anchor screws 660 and 665 may be set at different angles as shown-to take advantage of adjacent bone structures in providing more secure anchoring. While a single anchor screw may be used to secure each anchor base 645, 645′, the use of at least two anchor screws for each base ensures that the base will not spin about the screw axis when subjected to a moment load.

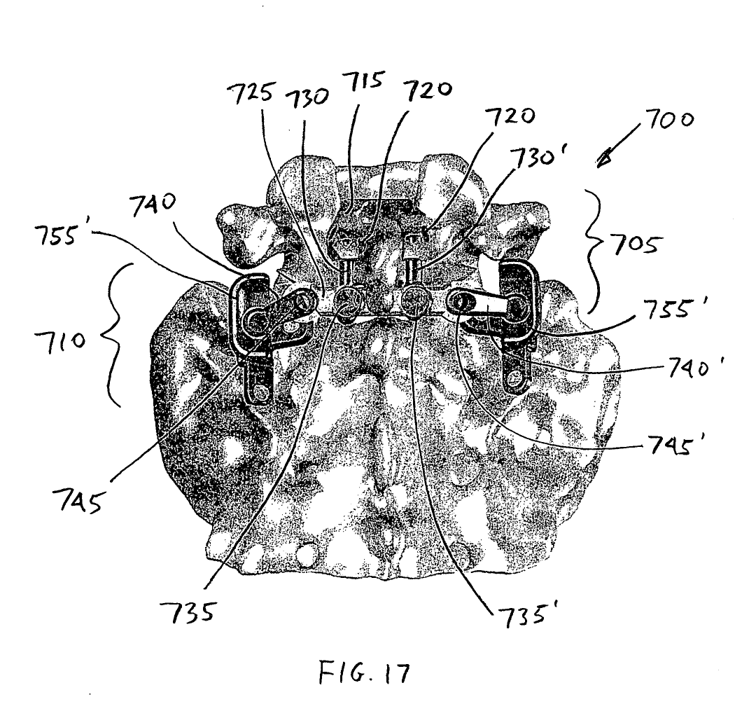

Referring now to FIG. 17, an isometric view of an implantable spinal arthroplasty device 700 is depicted with the device attached to the L5 vertebra and sacrum of a spine. Device 700 comprises a cephalad portion 705 and a caudal portion 710. Similar to the embodiment of FIGS. 15 and 16, cephalad portion 705 of device 700 is secured to a vertebra at least in part by hooking over an upper edge of its lamina and under the spinous process. Supralaminar hook body 715 may be formed as an integral unit having one or more hook members for extending from a cephalad surface to a caudal surface of the lamina. Anchor screws 720 may be provided for screwing into or against the lamina, spinous process, or a portion of the vertebra where the spinous process joins the lamina.

A spinous process clamp or crossbar 725 may be connected to the hook body 715 by a pair of connecting arms 730, 730′. Connecting arms 730, 730′ may be integrally formed with hook body 715, or provided as separate elements as shown to allow different length arms to be selected depending on the application and/or particular anatomy of the patient. Crossbar 725 may be secured to connecting arms 730, 730′ with fasteners 735, 735′. In this embodiment, fasteners 735, 735′ have external threads for engaging internal thread formed within bores in crossbar 725. As fasteners 735, 735′ are tightened down, they compress connecting arms 730, 730′ against a complementary shaped surface formed in crossbar 725, or against a lock insert such as crossbar lock 270 shown in FIGS. 8 and 9 and described above. Once cephalad portion 705 is assembled around the spinous process, it may be secured in place by tightening anchor screws 720 to drive crossbar 725 against the underside of the spinous process and hook portions of the hook body 715 against the underside of the lamina.

Outriggers 740, 740′ may be adjustably attached to the laterally outward ends of crossbar 725, such as with multi-axis joints 745, 745′. Cephalad bearing elements 750, 750′ may be mounted on the laterally outward ends of outriggers 740, 740′ such that they can be positioned to engage with mating caudle cups 755, 755′ and locked into position relative to crossbar 725 by multi-axis joints 745, 745′. Caudal cups 755, 755′ may be mounted to the sacrum or other vertebra in a manner similar to that shown in FIGS. 15 and 16 as described above, and/or as will be further described below.

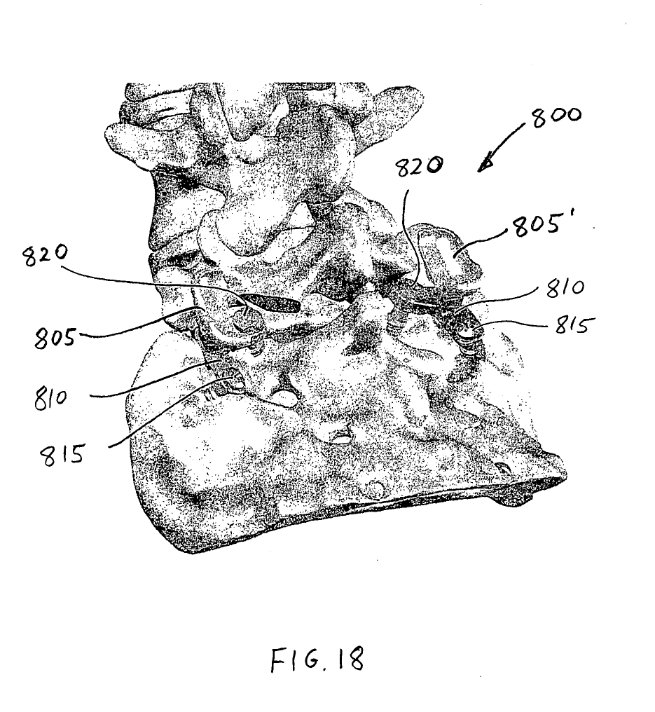

Referring to FIGS. 18-21, additional caudal anchoring details are shown. FIG. 18 shows a caudal bearing assembly 800 attached to a sacrum. Caudal assembly 800 may be used in conjunction with any of the cephalad assemblies disclosed or referred to herein to form artificial facet joints, particularly between an L5 vertebra and a sacrum. Caudal assembly 800 of this embodiment is formed by a pair of caudle cups 805, 805′ mounted on bases 810, 810′, which in turn are mounted to the spine by anchor screws 815 and 820. With this arrangement, bases 810, 810′ may be securely mounted either with bases contacting the spine, or with bases elevated from a surface of the spine as shown. Such a mounting system allows for greater flexibility in positioning the caudal cups 805, 805′ where they are desired in each procedure. In the embodiment shown, bases 810, 810′ are L-shaped, although other layouts may also be used.

As shown in FIG. 19, anchor screws 815 and 820 each have a pair of flats, 825 and 830 respectively, so that they may be turned with a wrench or other tool when inserting them into the bone. Each screw 815 and 820 also has a shoulder portion, 835 and 840 respectively, which provides a surface for supporting the bottom of a base 810, 810′. Anchor screw 815 includes an internally threaded bore 845 for receiving a screw 850. Screw 850 passes through hole 855 in a base 810, 810′ and into threaded bore 845 to tighten the base down onto shoulder portion 835. Anchor screw 820, on the other hand, has an externally threaded portion 860 extending above flats 830 such that it may be received in hole 865 in a base 810, 810′. Nut 870 may then be engaged with threaded portion 860 to tighten base 810, 810′ down onto shoulder portion 840 of anchor screw 820. With base 810, 810′ secured to anchor screws 815 and 820 in this manner, it may be securely positioned on or above the bone. The same type of screw 815, 820 or other screw may be used at all locations if desired.

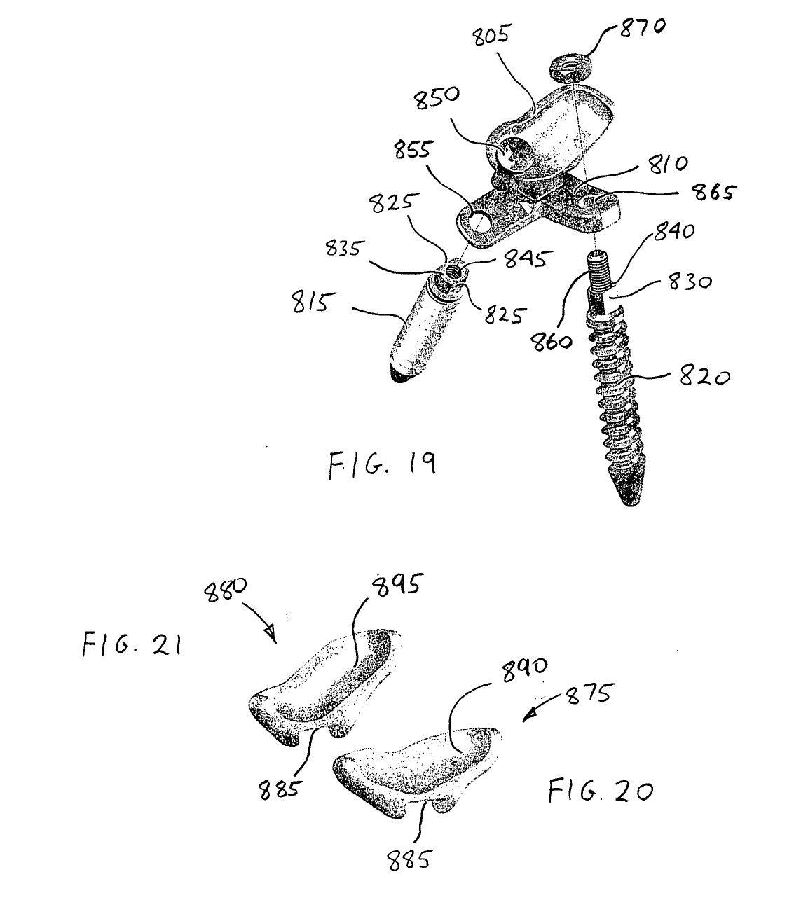

Referring to FIGS. 20 and 21, additional embodiments of caudal cups 875 and 880 are shown. Caudal cups 805, 805′ (both shown in FIG. 18), 875 and 880 may be attached to a base 810, 810′ using locking attachment surfaces 885 which mate with complementary shaped surfaces (not shown) located on bases 810, 810′. As seen in FIGS. 20 and 21, the bearing surfaces 890 and 895 of caudal cups 875 and 880 each have different configurations. For example, the bearing surface 890 of caudal cup 875 shown in FIG. 20 has more symmetrical anterior and posterior portions that are also inclined at shallower angles than the corresponding portions of bearing surface 895 of caudal cup 880 shown in FIG. 21. Many other variations of caudal cups may be provided in kit form for use in operating rooms. As will be appreciated by those skilled in the art, particular configurations of bearing surfaces may be selected to provide desired types and ranges of motion depending on the mounting position and other factors of each application.

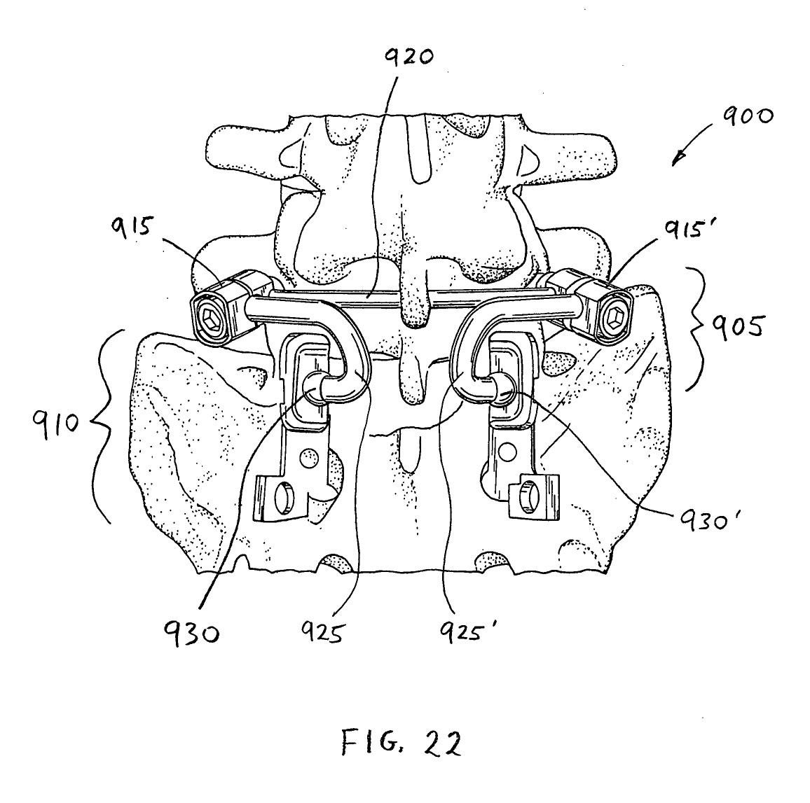

Referring to FIG. 22, an isometric view of an implantable spinal arthroplasty device 900 is depicted with the device attached to the L5 vertebra and sacrum of a spine. Device 900 comprises a cephalad portion 905 and a caudal portion 910. Cephalad portion 905 comprises a pair of anchor screw assemblies 915, 915′ which are attached by screws to the pedicles of the L5 vertebra in this embodiment. Anchor screw assemblies 915, 915′ are configured to receive ends of crossbar 920 which spans between the assemblies. Assemblies 915, 915′ are also configured to receive the proximal ends of cephalad bearing arms 925, 925′. Generally spherically shaped cephalad bearing members 930, 930′ may be provided at the distal ends of arms 925, 925′ for engaging with caudal bearing cups of caudal portion 910.

Crossbar 920 provides additional stability to anchor screw assemblies 915, 915′, such as securing them from rotational moments caused by forces on cephalad bearing members 930, 930′ at the distal ends of arms 925, 925′. Crossbar 920 may be straight, curved or have a complex shape to avoid adjacent anatomy, such as the spinous process of an adjacent vertebra. Anchor screw assemblies 915, 915′ may be constructed and mounted in a manner similar to that of anchors 210, 210′ shown in FIGS. 7-9 and described above. Further construction details of assemblies 915, 915′ may be obtained from U.S. patent application publication number 2005/0240265, published Oct. 27, 2005, for example in FIG. 16A-16F. Caudal portion 910 of device 900 may be constructed and mounted in a similar manner to the previous embodiments described above.

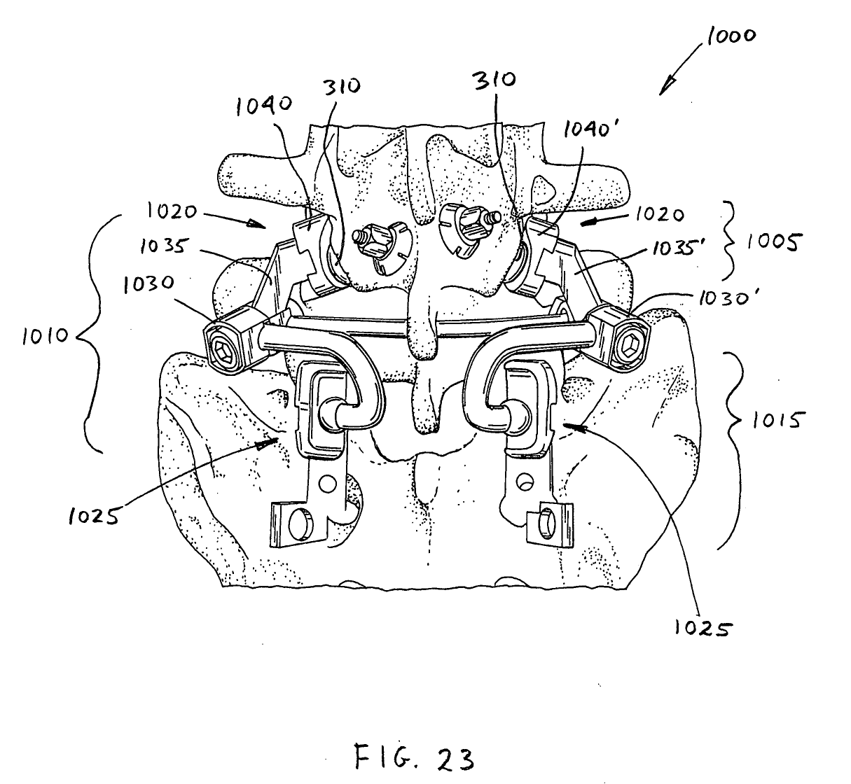

Referring to FIG. 23, an isometric view of a hybrid, multi-level implantable spinal arthroplasty device 1000 is depicted with the device attached to the L4 and L5 vertebrae and sacrum of a spine. While shown and described at this spinal location in this exemplary embodiment, device 1000 or a modified version thereof may be employed at other locations along the spine. Arthroplasty device 1000 comprises three main portions: a first or cephalad portion 1005, a second or middle portion 1010, and a third or caudal portion 1015. Cephalad portion 1005 and middle portion 1010 cooperate to form artificial facet joints 1020. In this embodiment, artificial joints 1020 replace the natural facet joints that have been removed from the L4-L5 level of the spine. Similarly, middle portion 1010 and caudal portion 1015 cooperate to form artificial facet joints 1025. In this embodiment, artificial joints 1025 replace the natural facet joints that have been removed from the L5-S1 level of the spine. In this manner, middle portion 1010 of device 1000 serves as the caudal portion of joints 1020 and the cephalad portion of joints 1025. As previously mentioned, this arrangement may be used at other levels of the spine, and may involve more than three vertebrae such as by adding additional sections.

In the embodiment shown in FIG. 23, cephalad portion 1005 comprises translaminar cephalad anchors 300. These may be the same as described above in conjunction with FIGS. 10-12. Alternatively, other translaminar cephalad anchor designs may be used, such as those shown in FIGS. 13 and 14. Anchors for cephalad bearing surfaces 310 may also be mounted to the pedicles of the vertebra, in this example vertebra L4.

Middle portion 1010 and caudal portion 1015 of device 1000 may be constructed in manner similar to that of device 900 shown in FIG. 22 and described above. However, in contrast to device 900, the anchor screw assemblies 1030, 1030′ of device 1000 include medially extending arms 1035, 1035′ for supporting caudal bearing members 1040, 1040′. Caudal bearing members 1040, 1040′ are positioned adjacent to and cooperate with cephalad bearing surfaces 310 to form artificial facet joints 1020.

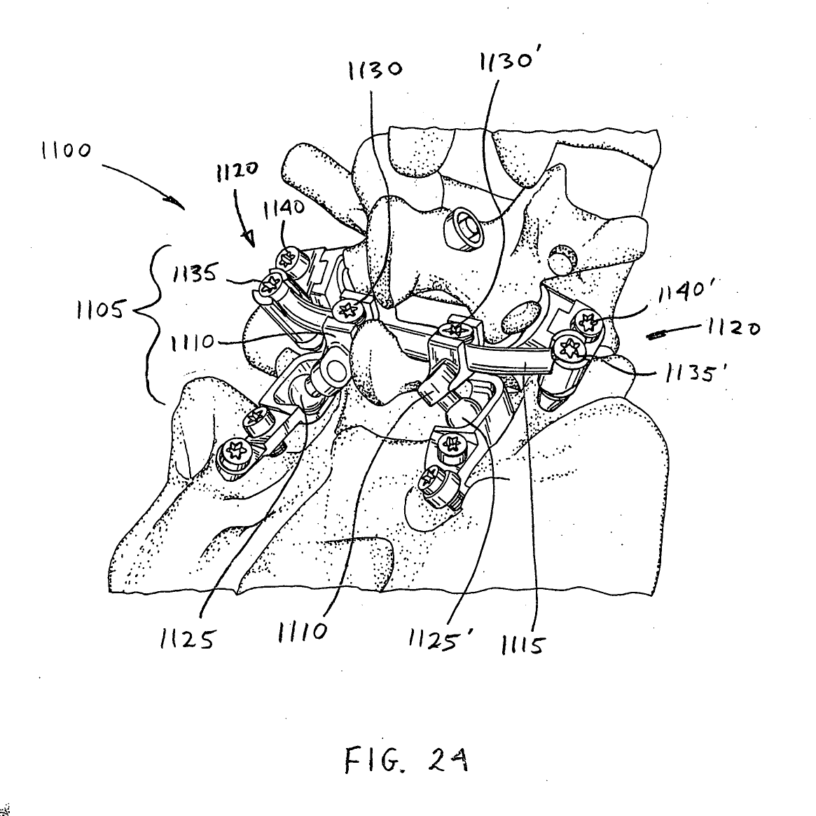

Referring to FIG. 24, an isometric view of another hybrid, multi-level implantable spinal arthroplasty device 1100 is depicted. Device 1100 is similar to device 1000 described above, and is also shown in an exemplary embodiment attached to the L4 and L5 vertebrae and sacrum of a spine, although it may be used at other locations of the spine. Middle portion 1105 of this embodiment has two main differences from the middle portion 1010 of device 1000 shown in FIG. 23. First, arms 1110, 1110′ are mounted to crossbar 1115 rather than directly to anchor screw assemblies 1120, 1120′. This arrangement allows arms 1110, 1110′ and the cephalad bearing member 1125, 1125′ mounted on their distal ends to be laterally adjusted by sliding arms 1110, 1110′ along crossbar 1115 and locking them in place with fasteners 1130, 1130′. Second, anchor screw assemblies 1120, 1120′ are provided with two pairs of screws 1135, 1135′ and 1140, 1140′. Screws 1135, 1135′ serve to secure the ends of crossbar 1115 to anchor screw assemblies 1120, 1120′. Screws 1140, 1140′ serve to secure anchor screw assemblies 1120, 1120′ to the vertebra. In this embodiment, screws 1140, 1140′ screw into the pedicles of the L5 vertebra. In other embodiments, multiple screws may be used to secure each anchor screw assembly to the vertebra.

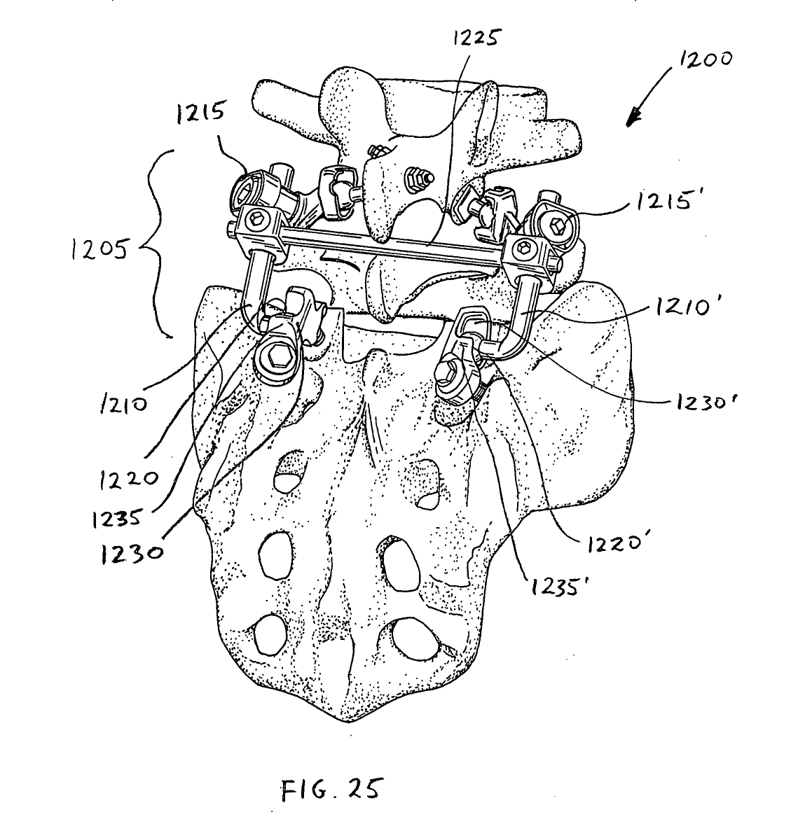

Referring to FIG. 25, an isometric view of yet another hybrid, multi-level implantable spinal arthroplasty device 1200 is depicted. Device 1200 is similar to devices 1000 and 1100 described above. Middle portion 1205 of device 1200 comprises cephalad bearing arms 1210, 1210′ which are adjustably secured at their proximal ends to anchor screw assemblies 1215, 1215′. Cephalad bearing members 1220, 1220′ are located at the distal ends of arms 1210, 1210′. Each end of crossbar 1225 is adjustably mounted to one of the arms 1210, 1210′. With this modular and adjustable arrangement, the position of cephalad bearing members 1220, 1220′ may be located precisely with respect to caudal bearing cups 1230, 1230′ and locked into place.

In the embodiment shown in FIG. 25, caudal bearing cups 1230, 1230′ face laterally outward rather than medially. Accordingly, unlike the previous example provided above, cephalad bearing arms 1210, 1210′ enter caudal cups 1230, 1230′ from the laterally outward side rather than from the medial side. Caudal cups 1230, 1230′ also have a G-shaped cross-section which creates an overhanging posterior surface 1235, 1235′ over a portion of caudal cups 1230, 1230′. Bearing surface 1235, 1235′ prevents movement of a vertebra, in this case L5, in a posterior direction when in its natural state. It is to be understood that features of any of the embodiments disclosed herein may be used in combination with other embodiments.

In some embodiments, the devices described herein and variations thereof may be implanted in conjunction with one or more artificial discs. In this way, correction of spinal degradation in one part of the spine does not cause spinal loads to be transmitted to adjacent spinal members that may also be failing. The various devices disclosed herein may be implanted before, after or in conjunction with disc replacement devices.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An implantable spinal arthroplasty device comprising:
a first portion adapted to engage a vertebra at the L5 level of the spine, the vertebra having two pedicles, a lamina and a spinous process; and
a second portion adapted to engage a portion of a sacrum,
wherein the first and the second portions cooperate to form at least one artificial facet joint between the L5 level and the sacrum.

2. The device of claim 1 wherein the first portion is configured to engage the pedicles of the L5 vertebra.

3. The device of claim 2 wherein the first portion comprises a first attachment member and a second attachment member, each attachment member configured to connect to one of the pedicles, the first portion further comprising a cross arm spanning between the first and the second attachment members.

4. The device of claim 1 wherein the first portion is configured to engage the lamina of the L5 vertebra.

5. The device of claim 4 wherein the first portion is configured to hook over an upper surface of the lamina.

6. The device of claim 1 wherein the first portion comprises two generally vertical members configured to be located on laterally opposite sides of a spinal process at the L5 level and a cross member configured to interconnect the generally vertical members and to be located beneath the spinal process.

7. The device of claim 1 wherein the first portion comprises at least one threaded fastener configured to penetrate the lamina.

8. The device of claim 1 wherein the first portion comprises at least one threaded fastener configured to press against an outer surface of the lamina.

9. The device of claim 1 further comprising a joint interconnecting the first and the second portions of the device, the joint have a generally ball-shaped portion and a cup portion, wherein the ball-shaped portion may slide relative to the cup portion.

10. A method of implanting a spinal arthroplasty device, the method comprising:
attaching the device of claim 1 to the spine of a patient.

11. The method of claim 10 further comprising replacing at least a portion of a natural disc located adjacent to L5 vertebra with an artificial disc implant.

12. A multi-level implantable spinal arthroplasty device comprising:
a first portion connectable to a first vertebra;
a second portion connectable to a second vertebra located beneath the first vertebra;
a third portion connectable to a third vertebra located beneath the second vertebra;
at least one upper artificial joint interconnecting and allowing relative movement between the first and the second portions; and
at least one lower artificial joint interconnecting and allowing relative movement between the second and the third portions.

13. The device of claim 12 wherein the first, the second and the third vertebrae are adjacent to one another.

14. The device of claim 13 wherein the first and the second vertebrae are at the L4 and L5 levels, respectively, and the third vertebra is part of the sacrum.

15. The device of claim 12 wherein the first portion is configured to attach to a lamina of the first vertebra.

16. The device of claim 12 wherein the second portion is configured to attach to at least one pedicle of the second vertebra.

17. The device of claim 12 wherein the third portion is configured to attach to at least one pedicle of the third vertebra.

18. The device of claim 12 wherein the first portion is configured to attach to a lamina of the first vertebra and the second portion is configured to attach to at least one pedicle of the second vertebra.

19. The device of claim 18 wherein the third portion is configured to attach to the sacrum.

20. The device of claim 12 wherein the first portion comprises two elongated bar elements, each element configured to pass diagonally through a lamina of the first vertebra.

21. The device of claim 20 wherein the at least one upper artificial joint comprises a convex member and a concave member, the two members being configured to inter-engage to provide relative movement between the first and the second portions, one of the two members being located on a lower end of one of the elongated bar elements.

22. The device of claim 21 wherein the at least one lower artificial joint comprises a generally ball-shaped portion and a cup portion, wherein the ball-shaped portion may slide relative to the cup portion

23. The device of claim 12 wherein the second portion comprises a first attachment member and a second attachment member, each attachment member configured to connect to a pedicle of the second vertebra, the second portion further comprising a cross arm spanning between the first and the second attachment members.

24. A method of implanting a multi-level spinal arthroplasty device, the method comprising:
attaching the device of claim 12 to the spine of a patient.

25. The method of claim 24 wherein the device is attached to three adjacent vertebrae.

26. The method of claim 25 wherein the first vertebra is at the L4 level, the second vertebra is at the L5 level and the third vertebra is part of the sacrum.

27. The method of claim 24 further comprising replacing at least a portion of a natural disc adjacent to any of the first, the second and the third vertebrae with an artificial disc implant.
